(19) United States
(12) Patent Application Publication
(10) Pub. No.: US 2026/0108204 A1
(43) Pub. Date: Apr. 23, 2026

(54) METHOD AND APPARATUS FOR IMPLEMENTING PREDICTION MODEL OF MUSCULOSKELETAL DISORDER

(71) Applicant: Alyce Healthcare Inc., Ulsan (KR)

(72) Inventors: Da kyum Kang, Ulsan (KR); Albert Myunki Han, Ellicott City, MD (US)

(21) Appl. No.: 18/931,171

(22) Filed: Oct. 30, 2024

(30) Foreign Application Priority Data
Oct. 23, 2024 (KR) 10-2024-0145372

(51) Int. Cl. A61B 5/00; A61B 5/11; G06T 7/00; G06T 7/246; G16H 10/60; G16H 20/30; G16H 50/20

(52) U.S. Cl. CPC A61B 5/45; A61B 5/1121; A61B 5/1128; A61B 5/7264; A61B 5/7275; A61B 5/742; G06T 7/0012; G06T 7/251; G16H 10/60; G16H 20/30; G16H 50/20; G06T 2207/30004

(57) ABSTRACT

A method for implementing a musculoskeletal disorder prediction model, performed by a musculoskeletal disorder prediction model implementation apparatus. The method includes: acquiring a user image which performs a pre-registered musculoskeletal test motion; extracting a feature point of a body part which performs the musculoskeletal test motion from the user image by using a motion recognition model implemented in advance; determining a range of motion ROM of a musculoskeletal system corresponding to the body part based on a movement trajectory of the extracted feature point of the body part; extracting a reference feature point of a pre-registered musculoskeletal normal range of motion image; comparing the movement trajectory of the extracted feature point of the body part and a movement trajectory of the reference feature point; and matching and storing a comparison result of the determined range of motion and the movement trajectory.

### CROSS-REFERENCE TO RELATED APPLICATIONS

This application claims the priority of Korean Patent Application No. 10-2024-0145372 filed on Oct. 23, 2024, in the Korean Intellectual Property Office, the disclosure of which is incorporated herein by reference.

### BACKGROUND

### Field

The present disclosure relates to a method and an apparatus for implementing a prediction model of the musculoskeletal disorder. More particularly, the present disclosure relates to a method and an apparatus for implementing a model of predicting the musculoskeletal disorder based on the joint range of motion measured through the artificial intelligence-based motion recognition.

### Description of the Related Art

In regard to a musculoskeletal system, due to an injury during exercise in which strong power is concentrated, such as tennis in addition to function degeneration due to aging, musculoskeletal disorder patients are caused. In addition, a working environment of modern workers, such as a PC office environment that repeatedly uses the same skeletal area, or loading and unloading, and delivery business in a distribution center, is exposed to musculoskeletal disorders.

In order to prevent such musculoskeletal disorders, accurate diagnosis of a current state and a risk of developing a disorder in the musculoskeletal system is required. In recent years, a system for diagnosing the musculoskeletal system by analyzing movements of main feature points of body joints is required. In particular, in order to increase the diagnosis accuracy of the system, an evaluation of a translocation motion such as an overhead squat is used.

However, in order to evaluate the translocation motion, the system should additionally include a 3-dimensional camera and equipment for measuring plantar pressure distribution, and there is a limit to diagnosing disorder developing possibilities in various joint portions of the body. In addition, as the progression of the musculoskeletal disorder, even though compensation movement can occur during a specific motion, there is a limit that a portion where the compensation movement actually occurs and the degree of compensation movement cannot be diagnosed.

The limit of the diagnosis makes a user in which the musculoskeletal system is degenerated or there is a high possibility of occurrence of the musculoskeletal disorder very difficult. That is, since accurate diagnosis for the current state is difficult, it is difficult for the user to appropriately respond to alleviation of a symptom of the musculoskeletal disorder or delay of a degeneration progress of the musculoskeletal system.

### SUMMARY

An object to be achieved by the present disclosure is to provide a method and an apparatus for extracting range of motion information of a joint through artificial intelligence-based motion recognition, and predicting a musculoskeletal disorder based thereon.

Further, another object to be achieved by the present disclosure is to provide a method and an apparatus for extracting compensation movement information through the artificial intelligence-based motion recognition, and predicting the musculoskeletal disorder based thereon.

Yet another object to be achieved by the present disclosure is to provide a method and an apparatus for implementing an occurrence prediction model of the musculoskeletal disorder by using the range of motion information of the joint and user information.

Further, still yet another object to be achieved by the present disclosure is to provide a method and an apparatus for guiding a prevention motion for alleviating a progress of the musculoskeletal disorder based on an occurrence risk of the musculoskeletal disorder.

The technical objects of the present disclosure are not restricted to the aforementioned technical objects, and other objects of the present disclosure, which are not mentioned above, will become more apparent to one of ordinary skill in the art to which the present disclosure pertains by referencing the detailed description of the present disclosure given below.

According to an exemplary embodiment of the present disclosure, provided is a method for predicting a musculoskeletal disorder based on motion recognition, which may include: acquiring a user image including a motion of a musculoskeletal body part; identifying a body part motion corresponding to at least one of pre-registered musculoskeletal test motions in the user image by using a motion recognition model implemented in advance; determining a range of motion ROM of a musculoskeletal system which performs the body part motion based on the identified body part motion; and predicting a disorder risk of the musculoskeletal system based on the determined range of motion ROM by using a musculoskeletal disorder prediction model implemented in advance.

In an exemplary embodiment, the identifying of the body part motion may include extracting a feature point of a body part including the musculoskeletal system by using the motion recognition model, tracking a movement trajectory of the extracted feature point for a predetermined time, and determining, when the tracked movement trajectory of the feature point is within a predetermined similarity range to a first test motion included in the musculoskeletal test motion, the body part motion as the first test motion.

In an exemplary embodiment, the determining of the body part motion as the first test motion may include determining a first feature point which moves within a predetermined range among the feature points as a center point, according to a tracking result of the movement trajectory, identifying a second feature point located from the first feature point by a predetermined distance, and identifying a movement trajectory in which the second feature point rotates based on the center point, and the determining of the range of motion of the musculoskeletal system may include generating at least one information of rotational distance information and rotational angle information of the second feature point around the center point based on the movement trajectory in which the second feature point rotates, and determining the musculoskeletal range of motion based on the generated information.

In an exemplary embodiment, the determining of the range of motion of the musculoskeletal system based on the generated information may include splitting the rotating movement trajectory of the second feature point into a plurality of predetermined same time-unit intervals, splitting into a plurality of intervals moved for predetermined same time intervals, determining, when an interval which deviates from a predetermined trajectory guide based on the first test motion is identified among the split intervals, the interval which deviates from the trajectory guide as a compensation movement interval, excluding at least a part of the compensation movement interval from the determined range of motion, and updating the range of motion from which the at least a part is excluded to the determined range of motion.

In an exemplary embodiment, the determining of the interval which deviates from the trajectory guide as the compensation movement interval may include determining, when a trajectory opposite to the trajectory guide of the second feature point is extracted, a first position in which an angular speed of the rotating movement trajectory becomes 0 as a start point of the compensation movement interval, determining a second position in which the angular speed becomes 0 as an end point of the compensation movement interval after the start point, and determining the compensation movement interval based on the start point and the end point.

In an exemplary embodiment, the identifying of the second feature point located from the first feature point by the predetermined distance may include identifying a third feature point forming a first angle based on a straight line including the first feature point and the second feature point within a predetermined first distance range from the first feature point among the tracked feature points, and identifying a fourth feature point forming a second angle based on the straight line within a predetermined second distance range from the first feature point among the tracked feature points, and the generating of at least one information of rotational distance information and rotational angle information of the second feature point around the center point may include generating distance change information and angle change information of the first angle between the third feature point and the first feature point while at least one information of the second feature point is generated, and generating distance change information and angle change information of the second angle between the fourth feature point and the first feature point while at least one information of the second feature point is generated.

In an exemplary embodiment, the determining of the range of motion of the musculoskeletal system based on the generated information may include, when among the distance change information and angle change information generated with respect to the third feature point and the fourth feature point, at least one change information which deviates from a predetermined trajectory guide based on the first test motion is extracted, determining the movement trajectory in which the second feature point rotates as the compensation movement interval while the change information which deviates from the trajectory guide is extracted, excluding at least a part of the compensation movement interval from the determined range of motion, and updating the range of motion from which the at least a part is excluded to the determined range of motion.

In an exemplary embodiment, the predicting of the disorder risk of the musculoskeletal system may include determining a pre-rated musculoskeletal disorder risk rating based on the determined range of motion, and predicting a change time of the determined risk rating based on the at least one extracted change information.

In an exemplary embodiment, the method may include: identifying an additional body part motion corresponding to a second test motion for additionally testing the musculoskeletal system which performs the first test motion among the pre-registered musculoskeletal test motions in the user image; and determining a range of motion of a musculoskeletal system which performs the additional body part motion based on the identified additional body part motion.

In an exemplary embodiment, the determining of the range of motion of the musculoskeletal system which performs the additional body part motion may include, when among the distance change information and angle change information generated with respect to the third feature point and the fourth feature point, at least one change information which deviates from a predetermined trajectory guide based on the second test motion is extracted, identifying at least one feature point of the third feature point and the fourth feature point as a feature point where a compensation movement occurs repeatedly, and the predicting of the disorder risk of the musculoskeletal system may include determining the pre-rated musculoskeletal disorder risk rating based on the determined range of motion, determining a risk weight based on the at least one extracted change information, and predicting a change time of the determined risk rating based on the risk weight.

In an exemplary embodiment, the identifying of the second feature point located from the first feature point by the predetermined distance may further include matching the straight line including the first feature point and the second feature point with a skeleton portion among elements constituting the musculoskeletal system, matching an area including the identified third feature point with a first muscle portion among factors of the musculoskeletal system, and matching an area including the identified fourth feature point with a second muscle portion among the factors of the musculoskeletal system.

In an exemplary embodiment, the method may further include: extracting a feature point of a body part including another musculoskeletal system connected to at least one element of the skeleton portion, the first muscle portion, and the second muscle portion; and setting an area including the feature point for another musculoskeletal system as a compensation movement spread area.

In an exemplary embodiment, the method may further include: identifying a body part motion corresponding to a test motion of another musculoskeletal system among the pre-registered musculoskeletal test motions in the user image by using the motion recognition model implemented in advance; determining a range of motion of another musculoskeletal system based on the corresponding body part motion; and predicting a disorder risk of another musculoskeletal system based on the determined range of motion and information on the compensation movement spread area by using the musculoskeletal disorder prediction model implemented in advance.

In an exemplary embodiment, the predicting of the disorder risk of the musculoskeletal system matched with the body part based on the determined range of motion by using the musculoskeletal disorder prediction model implemented in advance may include predicting the disorder risk of another musculoskeletal system based on a result of predicting the disorder risk of the musculoskeletal system and the information on the compensation movement spread area.

According to another exemplary embodiment of the present disclosure, provided is an apparatus for predicting a musculoskeletal disorder based on motion recognition, which may include: one or more processors; a network interface receiving a user image including a musculoskeletal body part motion; a memory loading computer programs executed by the processors; and a storage storing the computer programs.

In an exemplary embodiment, the computer program may include an operation of identifying a body part motion corresponding to at least one of pre-registered musculoskeletal test motions in the user image by using a motion recognition model implemented in advance, an operation of determining a range of motion ROM of a musculoskeletal system which performs the body part motion based on the identified body part motion, and an operation of predicting a disorder risk of the musculoskeletal system based on the determined range of motion ROM by using a musculoskeletal disorder prediction model implemented in advance.

According to an exemplary embodiment of the present disclosure, provided is a method for implementing a musculoskeletal disorder prediction model, which may include: acquiring a user image which performs a pre-registered musculoskeletal test motion; extracting a feature point of a body part which performs the musculoskeletal test motion from the user image by using a motion recognition model implemented in advance; determining a range of motion ROM of a musculoskeletal system corresponding to the body part based on a movement trajectory of the extracted feature point of the body part; extracting a reference feature point of a pre-registered musculoskeletal normal range of motion image; comparing the movement trajectory of the extracted feature point of the body part and a movement trajectory of the reference feature point; and matching and storing a comparison result of the determined range of motion and the movement trajectory.

In an exemplary embodiment, the comparing of the movement trajectory of the extracted feature point of the body part and the movement trajectory of the reference feature point may include first-splitting the movement trajectory of the feature point of the body part into a plurality of predetermined same time-unit intervals, second-splitting the movement trajectory of the reference feature point into the plurality of predetermined same time-unit intervals, first-identifying distance change information and angle change information of the first-split interval-specific extracted feature points, second-identifying distance change information and angle change information of the second-split interval-specific reference feature points, and matching and comparing the first-identified information and the second-identified information for each interval.

In an exemplary embodiment, the matching and comparing of the first-identified information and the second-identified information for each interval may include, when a difference between the first-identified information and the second-identified information exceeds a predetermined range according to the comparison result, determining that a compensation movement occurs in a matching interval in which the excess is confirmed.

In an exemplary embodiment, the extracting of the reference feature point may include identifying a plurality of feature points of the body part including the musculoskeletal system in the pre-registered musculoskeletal normal range of motion image by using a motion recognition model implemented in advance, and identifying a first feature point included in a first area and a second feature point included in a second area at both distal ends of the body part among the plurality of feature points, and the comparing of the movement trajectory of the extracted feature point of the body part and the movement trajectory of the reference feature point may include setting the first feature point which becomes a center of a rotating movement as a center point, and extracting target feature points matched with the first feature point and the second feature point, respectively among the extracted feature points of the body part, and first-comparing movement trajectories of the first feature point and the second feature point, and movement trajectories of the target feature points matched therewith, respectively.

In an exemplary embodiment, the extracting of the reference feature point may further include: identifying a third feature point forming a first angle based on a straight line including the first feature point and the second feature point within a predetermined first distance range from the first feature point, and identifying a fourth feature point forming a second angle based on the straight line within a predetermined second distance range from the first feature point, and the first-comparing of the movement trajectories of the first feature point and the second feature point, and the movement trajectories of the target feature points matched therewith, respectively may include determining that a compensation movement occurs when a difference for at least one of a distance and an angle of the movement trajectory exceeds a predetermined range according to a result of comparing the movement trajectories of the first feature point and the second feature point, and the movement trajectories of the target feature points matched therewith, respectively.

In an exemplary embodiment, the comparing of the movement trajectory of the extracted feature point of the body part and the movement trajectory of the reference feature point may further include extracting target feature points matched with the third feature point and the fourth feature point, respectively among the extracted feature points of the body part, and the determining that the compensation movement occurs may include second-comparing movement trajectories of the third feature point and the fourth feature point, and movement trajectories of target feature points matched with the third feature point and the fourth feature point, respectively, and the matching and storing of the comparison result of the determined range of motion and the movement trajectory may include matching and storing the determined range of motion, the first-compared result, and the second-compared result.

In an exemplary embodiment, the matching and storing of the comparison result of the determined range of motion and the movement trajectory may include collecting musculoskeletal diagnosis information of a user, which includes a response to a pain level of the user by performing the pre-registered musculoskeletal test motion, and matching and storing the range of motion, the comparison result of the movement trajectory, and the musculoskeletal diagnosis information of the user.

In an exemplary embodiment, the matching and storing of the range of motion, the comparison result of the movement trajectory, and the musculoskeletal diagnosis information of the user may include generating a first vector value set by embedding the matched and stored range of motion and the comparison result of the movement trajectory, generating a second vector value set by embedding the musculoskeletal diagnosis information of the user, for each predetermined cycle, and learning a relationship between the first vector value set and the second vector value set.

In an exemplary embodiment, the method may include: generating a musculoskeletal disorder prediction model based on the learning of the relationship between the first vector value set and the second vector value set; generating, as a latest range of motion for a musculoskeletal system of the user is determined, the musculoskeletal diagnosis information of the user based on the determined latest range of motion by using the generated musculoskeletal disorder prediction model; and predicting a disorder risk of the musculoskeletal system of the user based on the generated diagnosis information.

In an exemplary embodiment, the collecting of the musculoskeletal diagnosis information of the user may include generating a first vector value by performing a text analysis for a predetermined item in the collected diagnosis information, generating a second vector value by extracting a property of the user from the user image by using an analysis model implemented in advance, comparing the text-analyzed first vector value and the second vector value for the extracted property, generating a text based on the extracted second vector value when there is a difference which exceeds a predetermined range according to the comparison result, and setting the generated text in the predetermined item.

In an exemplary embodiment, the method may further include: generating an exercise program matched with a risk predicted for the musculoskeletal system; and providing the generated exercise program through a terminal of the user, and the exercise program may include exercise guide information for an area including the third feature point and an area including the fourth feature point where the compensation movement for the body part motion occurs.

According to another exemplary embodiment of the present disclosure, provided is an apparatus for implementing a musculoskeletal disorder prediction model, which may include: one or more processors; a network interface receiving a user image including a musculoskeletal body part motion; a memory loading computer programs executed by the processors; and a storage storing the computer programs, and the computer program may include an operation of acquiring a user image which performs a pre-registered musculoskeletal test motion, an operation of extracting a feature point of a body part which performs the musculoskeletal test motion from the user image by using a motion recognition model implemented in advance, an operation of determining a range of motion ROM of a musculoskeletal system corresponding to the body part based on a movement trajectory of the extracted feature point of the body part, an operation of extracting a reference feature point of a pre-registered musculoskeletal normal range of motion image, an operation of comparing the movement trajectory of the extracted feature point of the body part and a movement trajectory of the reference feature point, and an operation of matching and storing a comparison result of the determined range of motion and the movement trajectory.

According to an exemplary embodiment of the present disclosure, there is an effect in that a musculoskeletal disorder prediction model is implemented and provided. It is possible to predict the occurrence risk of a musculoskeletal disorder based on motion recognition by using the prediction model. Further, according to an exemplary embodiment, when implementing the prediction model, a user survey response type diagnosis result is reflected, so a prediction reliability can be enhanced.

As a result, according to the exemplary embodiment of the present disclosure, provided is an advantageous effect in that a user may prevent the musculoskeletal disorder before occurrence of the musculoskeletal disorder.

According to an exemplary embodiment of the present disclosure, there is an effect in that a plurality of different tests is performed for the same joint portion, so a musculoskeletal disorder occurrence prediction reliability is enhanced.

According to an exemplary embodiment of the present disclosure, there is an effect in that a motion guide for preventing a musculoskeletal disorder having a high occurrence risk is provided to prevent the musculoskeletal disorder, and lower the occurrence risk.

The effects of the present disclosure are not limited to the aforementioned effect, and other effects, which are not mentioned above, will be apparent to a person having ordinary skill in the art in the technical field of the present disclosure from the following disclosure.

### BRIEF DESCRIPTION OF THE DRAWINGS

The above and other aspects, features and other advantages of the present disclosure will be more clearly understood from the following detailed description taken in conjunction with the accompanying drawings, in which:

FIG. 1 is an exemplary diagram of a system for predicting a musculoskeletal disorder based on motion recognition according to an exemplary embodiment of the present disclosure;

FIG. 2 is a block diagram for an apparatus for predicting a musculoskeletal disorder based on motion recognition according to another exemplary embodiment of the present disclosure;

FIG. 3 illustrates an example of software for predicting a musculoskeletal disorder based on motion recognition according to another exemplary embodiment of the present disclosure;

FIG. 4 is a flowchart of a method for predicting a musculoskeletal disorder based on motion recognition according to yet another exemplary embodiment of the present disclosure;

FIGS. 5 and 6 illustrate examples for describing a joint range of motion referenced in some exemplary embodiments of the present disclosure;

FIGS. 7 to 12 illustrate examples for describing a compensation movement referenced in some exemplary embodiments of the present disclosure; and

FIG. 13 is a conceptual diagram of a prediction model according to still yet another exemplary embodiment of the present disclosure, and FIG. 14 is a flowchart of a method for implementing the prediction model of FIG. 13.

### DETAILED DESCRIPTION OF EXEMPLARY EMBODIMENTS

Hereinafter, preferred exemplary embodiments of the present disclosure will be described in detail with reference to the accompanying drawings. Advantages and features of the present disclosure, and methods for accomplishing the same will be more clearly understood from exemplary embodiments described in detail below with reference to the accompanying drawings. However, the present disclosure is not limited to the exemplary embodiments set forth below, and may be embodied in various different forms. The exemplary embodiments are just for rendering the disclosure of the present disclosure complete and are set forth to provide a complete understanding of the scope of the invention to a person with ordinary skill in the technical field to which the present disclosure pertains, and the present disclosure will only be defined by the scope of the claims. Throughout the specification, the same reference numerals denote the same elements.

Unless otherwise defined, all terms including technical and scientific terms used in this specification may be used as the meaning which may be commonly understood by the person with ordinary skill in the art, to which the present disclosure pertains. Terms defined in commonly used dictionaries should not be interpreted in an idealized or excessive sense unless expressly and specifically defined. It is also to be understood that the terms used herein are for the purpose of describing exemplary embodiments only and are not intended to limit the present disclosure. In this specification, the singular form also includes the plural form, unless the context indicates otherwise.

Hereinafter, in this specification, a system, a method, and an apparatus for predicting a musculoskeletal disorder based on motion recognition may be abbreviated as a musculoskeletal disorder prevention system, method, and apparatus, or a prediction system/method/apparatus, respectively.

Further, according to an exemplary embodiment of the present disclosure, the method and the apparatus for predicting a musculoskeletal disorder based on motion recognition may also be called a method and an apparatus for implementing a musculoskeletal disorder prediction model in terms of performing an exemplary embodiment for implementing the musculoskeletal disorder prediction model.

In this specification, terms such as “module”, “unit”, and “part” are one unit constituting software and/or hardware, and for example, “motion recognition unit” may mean a code bundle which performs a function in which the apparatus according to an exemplary embodiment of the present disclosure recognizes a body part motion of a user in a user image.

A hardware module/unit/part may be, for example, a hardware resource which is present for each processor for performing an operation of a specific function. The “module”, “unit”, and “part” may not only exist as a software module/unit/part or hardware module/unit/part, but may also mean a unit in which specific software and hardware are combined.

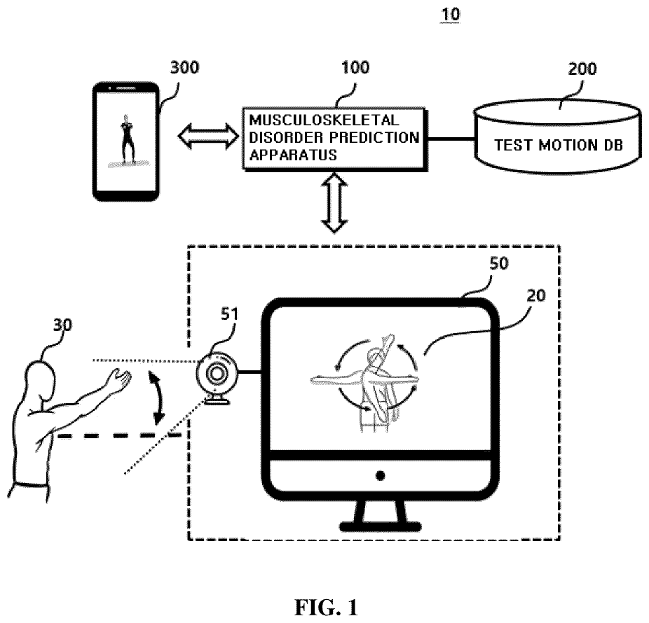

FIG. 1 is an exemplary diagram of a system for predicting a musculoskeletal disorder based on motion recognition according to an exemplary embodiment of the present disclosure.

Hereinafter, the system for predicting a musculoskeletal disorder according to the exemplary embodiment of the present disclosure may provide a service that skeleton-analyzes a body part motion of a user in real time by using artificial intelligence motion recognition technology to diagnose a musculoskeletal health condition corresponding to a body part of the user, and predict a risk of disorder occurrence.

Referring to FIG. 1, the musculoskeletal disorder prediction system 10 may include a musculoskeletal disorder prediction apparatus 100, a test motion DB 200, and a user terminal 300. The musculoskeletal disorder prediction apparatus 100, the test motion DB 200, and the user terminal 300 are computing devices that perform data communication with each other.

According to an exemplary embodiment, the musculoskeletal disorder prediction apparatus 100 may recognize the body part motion by using the artificial intelligence motion recognition technology, and measure a musculoskeletal range of motion ROM for the recognized body part motion. In the artificial intelligence motion recognition technology, the range of motion ROM widely known in the field to which the present disclosure pertains is a straight or curved distance at which a moving object may move in a state of being appropriately connected to another object. In particular, the musculoskeletal range of motion may mean, for example, a range in which a joint may move between ‘flexion’ and ‘extension’.

As another example, the range of motion may include a movable range of an ‘abduction motion’ meaning a movement which is far away from a center line of a body to the outside.

As yet another example, the range of motion may include a movable range of an ‘adduction motion’ meaning a movement which gets close to the center line of the body.

In general, each joint may have a normal range of motion, and there may be differences in the range of motion depending on the difference in age, sex, and flexibility for each person.

The musculoskeletal range of motion may be measured by using a goniometer and an inclinometer, and a range of motion measurement result may vary depending on the resistance of a user, patient. In particular, when there is a musculoskeletal injury, the range of motion may be limited due to pain, swelling, stiffness, etc.

According to the exemplary embodiment of the present disclosure, the musculoskeletal disorder prediction apparatus 100 compares the body part motion of the user recognized through motion recognition, and a pre-registered musculoskeletal test motion to determine whether the body part motion of the user matches the test motion. The motion recognition may be performed by using a motion recognition model based on at least one of artificial neural networks. For example, the artificial neural networks may include a convolution neural network CNN, or various transform models included in the CNN.

When the body part motion of the user matches the test motion, the musculoskeletal disorder prediction apparatus 100 may evaluate a risk based on the musculoskeletal range of motion of the user by using a musculoskeletal disorder prediction implemented in advance, and may also predict information on an occurrence possibility and/or an occurrence time of the musculoskeletal disorder of the user.

The test motion DB 200 may include at least one reference image for testing the musculoskeletal disorder of the user, and provide at least one reference image to the musculoskeletal disorder prediction apparatus 100. The reference image may include a musculoskeletal disorder test image of a trainer having a normal range of motion of the joint.

In an exemplary embodiment, the reference image may include test images of the trainer having various ages, different genders, and various body sizes. Here, the trainer may be a real person who pilots a musculoskeletal test motion, but the exemplary embodiment of the present disclosure is not limited thereto, but may include a virtual character implemented in graphics.

For example, the reference image stored in the test motion DB 200 may include at least one of motion images for a neer Impingement Test, a Hawkins-Kennedy Impingement Test, an empty can test, a drop arm test, a lift-off test, a straight leg raising SLR test, a crossed SLR test, and other tests widely known in the field to which the exemplary embodiment of the present disclosure pertains.

According to an exemplary embodiment, the musculoskeletal disorder prediction system 10 may also further include a display 50 and a camera 51 in order to perform the method according to the exemplary embodiment of the present disclosure.

In an exemplary embodiment, a user 30 may perform the body part motion according to a musculoskeletal test motion 20 provided through the display 50. The motion 20 may be a motion included in at least one image among the reference images.

The musculoskeletal disorder prediction apparatus 100 may acquire the body part motion of the user by the camera 51, and perform the motion recognition to determine a musculoskeletal range of motion of the user 30.

According to the exemplary embodiment of the present disclosure, the musculoskeletal disorder prediction apparatus 100 may receive diagnosis information for a musculoskeletal system of the user 30 from a medical institution server that performs a diagnosis of the user 30, or the user terminal 300. For example, the diagnosis information may include physical information of the user 30, such as gender, age, and obesity, and information on a pain level recognized by the user 30 and other medical histories when performing a motion of testing a musculoskeletal range of motion ROM.

Meanwhile, the tested range of motion may include at least one of a passive range of motion PROM in which a therapist or equipment moves the joint without user's efforts, an active assisted range of motion AAROM in which the user performs the exercise by using the muscles around the joint, but an assistance of the therapist or equipment is required, and an active range of motion AROM which is a range in which the user may move the muscles around the joint of himself/herself without the assistance of the therapist or equipment.

Further, the diagnosis information may include information in an electronic document format generated based on a response of the user 30 to a survey provided by a medical institution, and/or a diagnosis of a doctor.

In yet another exemplary embodiment, the musculoskeletal disorder prediction apparatus 100 may generate exercise guide information for alleviating or preventing the musculoskeletal disorder for the user 30, and provide the generated exercise guide information to the user terminal 300.

In FIG. 1, it is illustrated that the user terminal 300 is one component of the musculoskeletal disorder prediction system 10, but according to another exemplary embodiment, the musculoskeletal disorder prediction system 10 may be configured except for the user terminal 300, and the musculoskeletal disorder prediction apparatus 100 is integrated with the test motion DB 200 to be configured as one apparatus.

The musculoskeletal disorder prediction apparatus 100 may control functions and motions of the test motion DB 200, the display 50, the camera 51, and the user terminal 300 in executing musculoskeletal disorder prediction software according to the exemplary embodiment of the present disclosure.

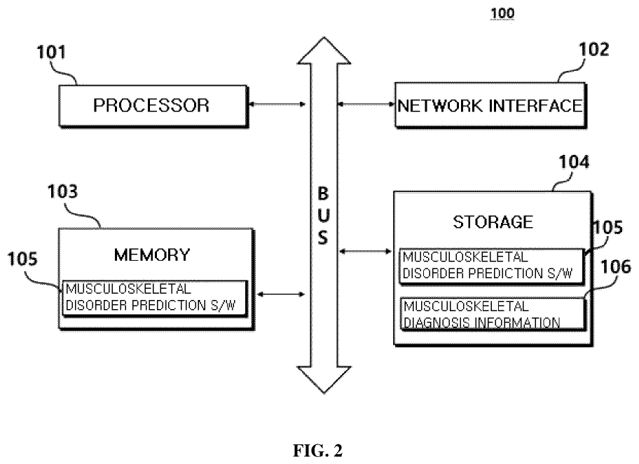

FIG. 2 is a block diagram for an apparatus for predicting a musculoskeletal disorder based on motion recognition according to another exemplary embodiment of the present disclosure.

The apparatus for predicting a musculoskeletal disorder based on motion recognition may include one or more processors 101, a network interface 102 for receiving an image of a body part motion of a user, which is photographed by a camera, a memory 103 for loading a computer program 105 executed by the processor 101, and a storage 104 for storing the computer program 105.

The processor 101 controls an overall motion of each component of the musculoskeletal disorder prediction apparatus 100. The processor 101 may be configured to include a central processing unit CPU, a micro processor unit MPU, a micro controller unit MCU, an application processor AP, or any type of processor well-known in the technical field of the present disclosure. Further, the processor 101 may perform an operation of at least application and/or program for executing the method according to the exemplary embodiments of the present disclosure.

The network interface 102 supports wired/wireless Internet communication of the musculoskeletal disorder prediction apparatus 100. Further, the network interface 102 may also support various communication schemes in addition to the Internet which is a public communication network. Further, the network interface 102 may also provide connections with the test motion DB 200, the user terminal 300, and/or the medical institution server. To this end, the network interface 102 may be configured to include at least one of a communication module and a connection terminal well-known in the technical field of the present disclosure.

According to the exemplary embodiment of the present disclosure, the network interface 102 may also form an interface with the artificial neural network well-known in the technical field to which the present disclosure pertains.

According to an exemplary embodiment, the musculoskeletal disorder prediction apparatus 100 may identify the body part motion of the user, and determine whether the identified body part motion matches the musculoskeletal test motion, by using a pre-trained motion recognition model in the artificial neural network.

The memory 103 stores various types of data, commands, and/or information. The memory 103 may load one or more programs 105 from the storage 140 in order to execute the exemplary embodiments of the present disclosure. In FIG. 2, the memory 130 may be, for example, a RAM.

The storage 104 may store the one or more programs 105, and musculoskeletal diagnosis information 106. In FIG. 2, as an example of the one or more programs 105, musculoskeletal disorder prediction software 105 is illustrated.

In an exemplary embodiment, the musculoskeletal diagnosis information 106 may include the diagnosis information for the musculoskeletal system of the user 30 received from the medical institution server that performs the diagnosis of the user 30 or the user terminal 300 referenced in FIG. 1.

In another exemplary embodiment, the musculoskeletal diagnosis information 106 may also include musculoskeletal diagnosis information generated based on a measurement result for the musculoskeletal range of motion of the user.

The storage 104 may be configured to include a nonvolatile memory such as a read only memory ROM, an erasable programmable ROM EPROM, an electrically erasable programmable ROM EEPROM, a flash memory or the like, a hard disk, a removable disk, or any type of computer-readable recording medium well-known in the art to which the present disclosure pertains.

Further, in FIG. 2, a case where the storage 104 is one component of the musculoskeletal disorder prediction apparatus 100 is illustrated, but the exemplary embodiment of the present disclosure is not limited thereto, and may also exist as an external component of the musculoskeletal disorder prediction apparatus 100 like a cloud connected by the network.

In the musculoskeletal disorder prediction software 105, according to the exemplary embodiment of the present disclosure, the processor 101 of the musculoskeletal disorder prediction apparatus 100 executes each operation to carry out the musculoskeletal disorder prediction method.

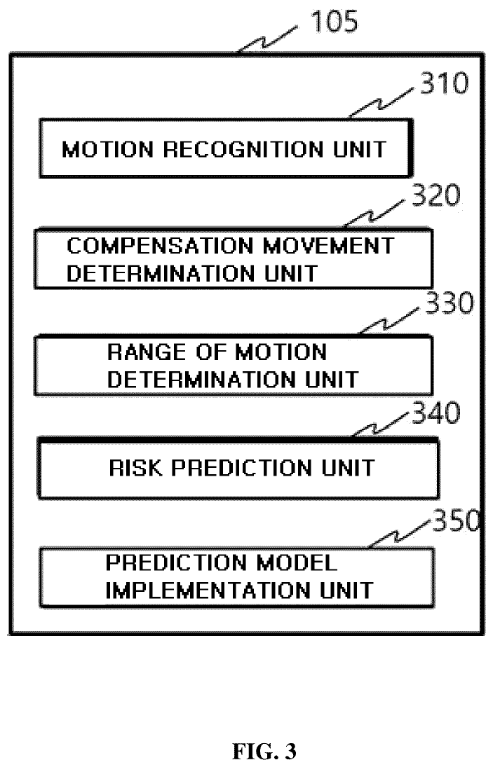

FIG. 3 illustrates an example of software for predicting a musculoskeletal disorder based on motion recognition according to another exemplary embodiment of the present disclosure. Referring to FIG. 3, the musculoskeletal disorder prediction apparatus 100 may execute software 105 for predicting the musculoskeletal disorder.

The software 105 may be configured to include a plurality of ‘units’ as function units. In FIG. 3, the software 105 may include a motion recognition unit 310, a compensation movement determination unit 320, a range of motion determination unit 330, a risk prediction unit 340, and a prediction model implementation unit 350. Hereinafter, a motion of each component of the software 105 is a motion which the musculoskeletal disorder prediction apparatus 100 performs by an operation of each component by the processor 101, but for convenience of description, it is described that each component operates.

As an example, the motion recognition unit 310 may determine whether the body part motion of the user is a pre-registered musculoskeletal test motion. To this end, the motion recognition unit 310 may use a pre-trained artificial intelligence motion recognition model stored in the storage 104 or connected through the network interface 102.

As an example, when the motion of the body part of the user identified by the motion recognition unit 310 satisfies a predetermined condition, for example, when a distance and/or angle information of at least a partial section of a movement trajectory of the body part is within a predetermined similarity range to the test motion, the motion of the body part of the user may be determined as the musculoskeletal test motion. To this end, the motion recognition unit 310 may extract a feature point of the user's body part within a user image, and extract a feature point of a body part corresponding to the test motion from the reference image.

The compensation movement determination unit 320 may determine whether the compensation movement occurs in the body part motion of the user who performs the musculoskeletal test motion. Here, the compensation movement means that a targeted motion is performed by using a power or a motion of another organ when it is difficult to perform the targeted motion due to disorder, degeneration, etc., of the skeleton and/or muscle in operating the musculoskeletal system. The compensation movement occurs when a motion is performed which becomes a burden on a predetermined element of the musculoskeletal system or another organ due to the abnormality of the musculoskeletal system.

According to the exemplary embodiment of the present disclosure, the compensation movement determination unit 320 may determine whether the compensation movement occurs in the body part motion of the user. Further, a body area in which the compensation movement occurs, for example, a muscle part connected to the musculoskeletal system may also be determined.

The range of motion determination unit 330 may measure the musculoskeletal range of motion based on the movement trajectory of the feature point extracted by the motion recognition unit 310. In particular, according to the exemplary embodiment of the present disclosure, the range of motion determination unit 330 may remove the movement trajectory by the compensation movement from the movement trajectory of the user body part motion in measuring the range of motion. As a result, it becomes possible to measure a precise musculoskeletal range of motion.

The risk prediction unit 340 may predict the occurrence risk of the musculoskeletal disorder of the user by considering various factors including the musculoskeletal range of motion of the user, user diagnosis information, user body information, an exercise amount of the user, etc. For data-based prediction, according to the exemplary embodiment of the present disclosure, the musculoskeletal disorder prediction model may be implemented in advance.

The prediction model implementation unit 350 may be implemented by learning the relationship of embedding result vector values of respective data items by embedding various data required for measuring the musculoskeletal disorder.

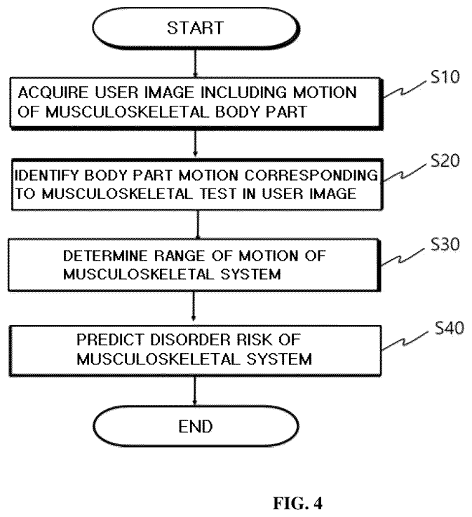

FIG. 4 is a flowchart of a method for predicting a musculoskeletal disorder based on motion recognition according to yet another exemplary embodiment of the present disclosure.

Each step of FIG. 4 is performed by the musculoskeletal disorder prediction apparatus 100, and specifically, each step is executed as the processor 101 of the musculoskeletal disorder prediction apparatus 100 performs an operation for each component of the software 105.

Referring to FIG. 4, the musculoskeletal disorder prediction apparatus 100 may acquire an image including a motion of a body part of a user, in S10. For example, the musculoskeletal disorder prediction apparatus 100 may receive the image through the camera 51 referenced in FIG. 1.

The musculoskeletal disorder prediction apparatus 100 may identify a body part motion corresponding to a predetermined test motion among pre-registered musculoskeletal test motions in an image including the motion of the body part of the user input by using a pre-trained artificial intelligence motion recognition model, in S20.

As an example, the identification of the body part motion may be performed by extracting a feature point by a pre-trained motion recognition model. The musculoskeletal disorder prediction apparatus 100 may extract a feature point for a body joint part and/or a body area of a user within the image of the body part motion of the user, and determine whether at least a partial motion of the user matches a predetermined test motion by using at least some information of a movement distance of coordinates of the extracted feature point, a direction, a distance between respective feature points, and/or angle.

Specifically, the musculoskeletal disorder prediction apparatus 100 may extract a feature point of a body part including a musculoskeletal system by using a motion recognition model.

In an exemplary embodiment, the musculoskeletal disorder prediction apparatus 100 may track a movement trajectory of the extracted feature point for a predetermined time. That is, a feature point which moves in time series according to the body part motion of the user generates the movement trajectory, and the musculoskeletal disorder prediction apparatus 100 tracks the movement trajectory.

When the tracked movement trajectory of the feature point is within a predetermined similar range to a predetermined test motion included in a musculoskeletal test motion, the musculoskeletal disorder prediction apparatus 100 may determine the body part motion as the predetermined test motion.

The musculoskeletal disorder prediction apparatus 100 may determine a range of motion ROM of a musculoskeletal system which performs the body part motion based on the identified body part motion, in S30.

At this time, the musculoskeletal system may include organs constituting a body, such as a skeleton and muscle included in the body part.

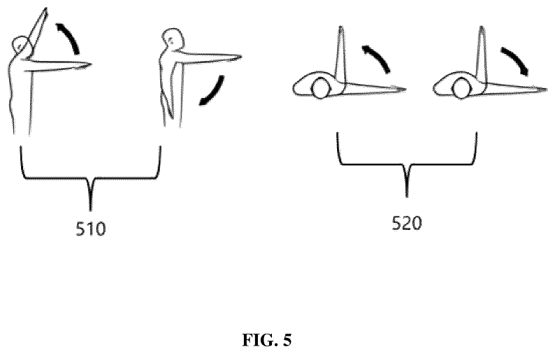

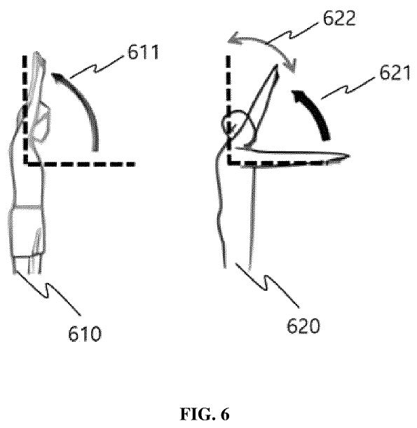

FIGS. 5 and 6 illustrate examples for describing a joint range of motion referenced in some exemplary embodiments of the present disclosure.

In particular, in FIG. 5, a musculoskeletal range of motion test motion at a right shoulder portion of the user is illustrated. A motion 510 is a test motion for upward and downward range of motion of a right shoulder, and a motion 520 is a motion of testing the range of motion by an abduction motion of getting close to the center line of the body of the user and an abduction motion of being far away from the center line.

Referring to FIG. 6, a motion 610 indicates a normal range of motion, and a motion 620 indicates an example of a motion in which there is a limit in a shoulder musculoskeletal range of motion. As an example, the motion 610 may be one scene of the reference image, and the motion 620 may be one scene of the body part motion of the user.

As the right arm of a trainer rotates and moves upward, the motion 610 generates a movement trajectory 611. In contrast, the user does not follow the movement trajectory 611, and generates a movement trajectory 621 through the motion 620.

In such a situation, the musculoskeletal disorder prediction apparatus 100 determines that the movement trajectory of the feature point tracked in the motion 620 is within a predetermined similar range to a predetermined test motion 610 included in the musculoskeletal test motion to determine the body part motion 620 as at least a part of the predetermined test motion 610.

Next, the musculoskeletal disorder prediction apparatus 100 may determine the shoulder musculoskeletal range of motion of the user. Specifically, the musculoskeletal disorder prediction apparatus 100 may identify time-series information for a distance and/or an angle of the movement trajectory 621, which does not follow the movement trajectory 611.

In an exemplary embodiment, the musculoskeletal disorder prediction apparatus 100 may identify a residual movement trajectory 622 based on the movement trajectory 611.

The musculoskeletal disorder prediction apparatus 100 may predict the musculoskeletal disorder risk based on the determined range of motion by using a pre-implemented musculoskeletal disorder prediction model, in S40. The risk may be rated information or a quantified score, and may include prediction information for the type of disorder, an occurrence time of the disorder, etc.

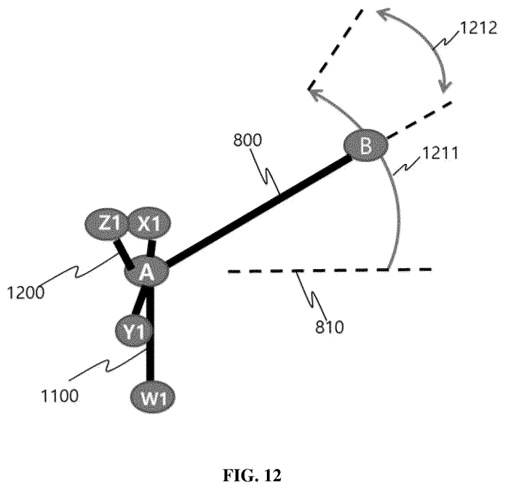

A detailed description of the musculoskeletal disorder prediction model will be made later in a description of FIG. 12.

Meanwhile, the musculoskeletal disorder prediction apparatus 100 may determine the musculoskeletal range of motion based on the motion 610 and the motion 620, but the range of motion determined by such a scheme may have an error by the compensation movement.

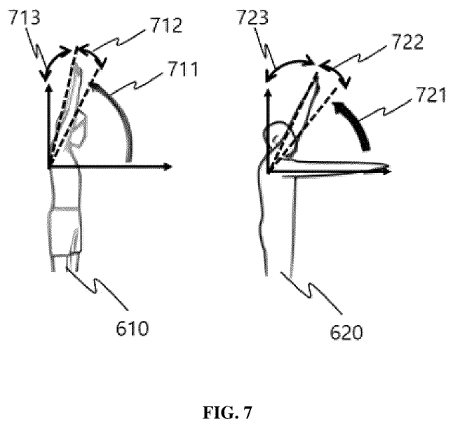

In order to resolve such an error, hereinafter, a method for removing a range of motion increased by the compensation movement according to an exemplary embodiment of the present disclosure will be described with reference to FIGS. 7 to 11.

FIGS. 7 to 12 illustrate examples for describing a compensation movement referenced in some exemplary embodiments of the present disclosure.

Referring to FIGS. 7 to 10, the musculoskeletal disorder prediction apparatus 100 may determine, as a center point, a first feature point which moves within a predetermined range among feature points according to a tracking result for the movement trajectory of the feature point extracted during performing the motion 620 of FIG. 7. Here, the predetermined range may be set to a range for extracting a center point in which a movement range is insignificant.

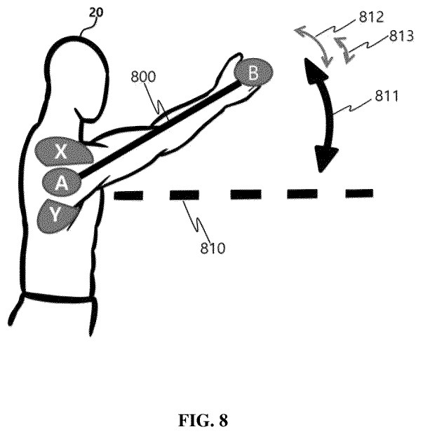

Referring to FIG. 8, the musculoskeletal disorder prediction apparatus 100 may determine the first feature point as the center point A in the motion of the body part which rotates and moves. The musculoskeletal disorder prediction apparatus 100 may identify a second feature point B located from the first feature point at a predetermined distance, and identify a movement trajectory 811 in which the second feature point B rotates around the center point A.

For example, the center point A indicates a shoulder skeleton, and the second feature point B indicates a hand end or wrist joint connected from the shoulder skeleton, and measuring a range of motion of the shoulder skeleton, and the center point A and the second feature point B may form a straight line 800, and an area X and an area Y adjacent to the center point A may be muscle areas that support the shoulder skeleton.

In an exemplary embodiment, the musculoskeletal disorder prediction apparatus 100 may identify a predetermined diagnosis target musculoskeletal system for the test motion identified as matching the body part motion of the user. For example, when the identified test motion is an upward rotating movement of the shoulder joint, the musculoskeletal disorder prediction apparatus 100 identifies a pre-registered diagnosis target musculoskeletal system matched with the test motion to extract the straight line 800, the muscle area X, and the muscle area Y corresponding to a skeleton.

In addition to identifying the pre-registered diagnosis target for the identified test motion, according to an additional exemplary embodiment of the present disclosure, the musculoskeletal disorder prediction apparatus 100 may also extract the muscle area X and the muscle area Y based on user's physical information identified by feature point extraction in the image.

As an example, the musculoskeletal disorder prediction apparatus 100 may also determine multiple feature point concentration areas in which trajectory movement occurs as the muscle area by performing the body part motion in addition to the area identified as the skeleton in the user image.

The musculoskeletal disorder prediction apparatus 100 may generate at least one information of rotational distance information and rotational angle information of the second feature point B around the center point A based on the movement trajectory 811 in which the second feature point B rotates, and determine the musculoskeletal range of motion based thereon.

However, when determining the range of motion, there is a problem of error occurrence due to the compensation movement described above, so the musculoskeletal disorder prediction apparatus 100 may determine the compensation movement, and remove the error.

That is, in FIG. 8, after the movement trajectory 811, a trajectory 812 in which the arm moves upwards, and then returns may occur by the compensation movement. The movement trajectory 811 and the trajectory 812 generate an overlapped trajectory 813. In an area where the overlapped trajectory 813 or the movement trajectory 811 and the trajectory 812 are in contact with each other, the user continues to perform the motion in a limit of the range of motion, so the compensation movement may occur. That is, the overlapped trajectory 813 may be determined as a trajectory corresponding to the compensation movement.

In an exemplary embodiment, the musculoskeletal disorder prediction apparatus 100 may remove a cause by the compensation movement by removing the trajectory 813 from the movement trajectory 811, and measure a precise range of motion.

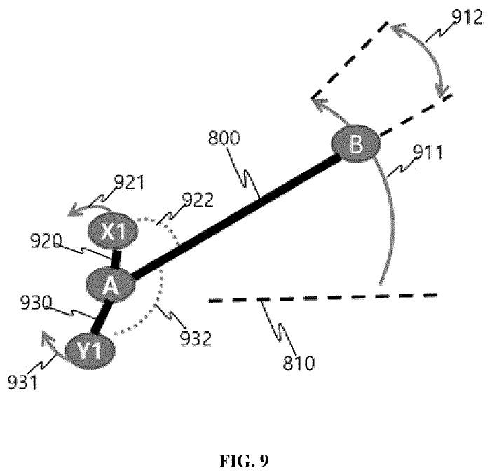

FIG. 9 illustrates an example in which the user's body part motion of FIG. 8 is expressed as a feature point connection structure. In particular, the feature point connection structure is illustrated as a structure in which a feature point X1 and a feature point Y1 included in the muscle area X and the muscle area Y adjacent to the center point A are connected to the center point A indicating the center point of the shoulder skeleton in a straight line.

In an exemplary embodiment, the musculoskeletal disorder prediction apparatus 100 may extract a plurality of feature points A, B, X1, and Y1 from the image including the user's body part motion.

In an exemplary embodiment, the musculoskeletal disorder prediction apparatus 100 may identify a third feature point X1 forming a first angle 922 with the center point A based on the straight line 800 formed by the second feature point B in a distance 920 from the center point A within a predetermined first distance range.

In an exemplary embodiment, the musculoskeletal disorder prediction apparatus 100 may identify a fourth feature point Y1 forming a second angle 932 based on the straight line 800 in a distance 930 from the center point A within a predetermined second distance range.

In an exemplary embodiment, the musculoskeletal disorder prediction apparatus 100 may generate distance 920 change information and angle change information of the first angle 922 between the third feature point X1 and the center point A while rotational distance change information and/or rotational angle change information generated with rotating movement of the second feature point B around the center point A are/is generated.

Further, the musculoskeletal disorder prediction apparatus 100 may also generate distance 930 change information and angle change information of the second angle 932 between the third feature point Y1 and the center point A by the same scheme.

According to an exemplary embodiment of the present disclosure, the musculoskeletal disorder prediction apparatus 100 may determine, as a movement trajectory tracking target, the feature point X1 among the plurality of extracted feature points within the muscle area X identified by the feature point extraction in the acquired image, and the feature point Y1 among the plurality of extracted feature points in the muscle area Y.

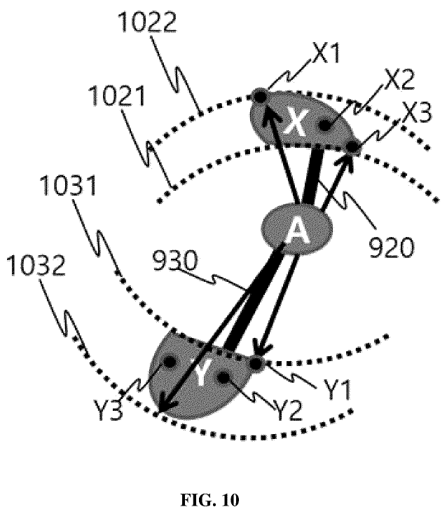

Referring to FIG. 10, the musculoskeletal disorder prediction apparatus 100 identifies the diagnosis target musculoskeletal system based on the identified test motion to extract the skeleton, the muscle area X, and the muscle area Y constituting the musculoskeletal system.

The musculoskeletal disorder prediction apparatus 100 may extract the plurality of feature points on the muscle area X and the muscle area Y, which include the center point A in the user image in which the body part motion matched with the test motion is performed.

In FIG. 10, in particular, a case where the feature point X1, the feature point X2, and the feature point X3 are extracted in the muscle area X, and the feature point Y1, the feature point Y2, and the feature point Y3 are extracted in the muscle area Y is illustrated as an example.

In an exemplary embodiment, the musculoskeletal disorder prediction apparatus 100 may determine, as the movement trajectory tracking target, at least one of the feature points which belong to the muscle area X as the feature point from the center point A within the first distance range.

For example, a distance 1021 and a distance 1022 may be set to the first distance range, and the musculoskeletal disorder prediction apparatus 100 may set a distance range including the identified muscle area X as the first distance range.

In an exemplary embodiment, the musculoskeletal disorder prediction apparatus 100 may determine, as the movement trajectory tracking target, at least one of the feature points which belong to the muscle area Y as the feature point from the center point A within the second distance range.

For example, a distance 1031 and a distance 1032 may be set to the second distance range, and the musculoskeletal disorder prediction apparatus 100 may set a distance range including the identified muscle area Y as the first distance range.

In an exemplary embodiment, the musculoskeletal disorder prediction apparatus 100 may determine, as a tracking target feature point, a feature point having a largest movement trajectory in each muscle area for a predetermined first time for which the user performs the body part motion according to the test motion among the extracted feature point X1, feature point X2, and feature point X3, and the extracted feature point Y1, feature point Y2, and feature point Y3, after the predetermined first time.

In another exemplary embodiment, the pre-registered diagnosis target musculoskeletal information matched with the test motion may include tracking candidate feature point information, and the musculoskeletal disorder prediction apparatus 100 may also determine the tracking target feature point in the user image matched with the test motion based on the tracking candidate feature point information.

Referring back to FIG. 4, the musculoskeletal disorder prediction apparatus 100 may determine the musculoskeletal range of motion based on the generated distance change information and/or angle change information.

In an exemplary embodiment, the musculoskeletal disorder prediction apparatus 100 may extract at least one change information which deviates from a predetermined trajectory guide based on a test motion which is being performed among distance change information and angle change information generated with respect to the third feature point X1 and the fourth feature point Y1.

Here, the predetermined trajectory guide as information on a movement trajectory of each reference feature point extracted from the reference image for each test motion includes reference information for the movement trajectory of the feature point extracted from the user image, which corresponds to the reference feature point.

According to an exemplary embodiment of the present disclosure, when a trajectory in which musculoskeletal-related feature points of the reference image move, and a movement trajectory of the musculoskeletal-related feature point extracted from the user image are compared, which exceed a predetermined range, the musculoskeletal disorder prediction apparatus 100 may determine that the compensation movement occurs.

In an exemplary embodiment, the musculoskeletal disorder prediction apparatus 100 may determine an interval 812 in which the change information which deviates from the trajectory guide is extracted in the movement trajectory 811 in which the second feature point is rotated as a compensation movement interval.

As an example, when a movement trajectory 921 of the third feature point X1 and a movement trajectory 931 of the fourth feature point Y1 show a difference which exceeds an allowable range from movement trajectories of reference feature points corresponding thereto on the predetermined trajectory guide, the musculoskeletal disorder prediction apparatus 100 may determine that the compensation movement occurs in an interval in which such a difference occurs. That is, as the compensation movement, when the user performs an unreasonable motion, a shape of the muscle area X to which the third feature point X1 belongs and/or the muscle area Y to which the fourth feature point Y1 belongs may show a difference compared to the motion in the normal range of motion.

As a result, the movement trajectory 921 of the third feature point X1 and the movement trajectory 931 of the fourth feature point Y1 may also represent abnormal trajectories, and in this case, the musculoskeletal disorder prediction apparatus 100 may determine an interval in which the abnormal trajectory appears as the interval 912 in which the compensation movement occurs.

The musculoskeletal disorder prediction apparatus 100 may exclude at least a part of the compensation movement interval 912 from an initially determined range of motion. Based thereon, the musculoskeletal disorder prediction apparatus 100 may update the initially determined range of motion in step S30 of FIG. 4 to a range of motion in which at least a part of the compensation movement interval is excluded.

As a result, in step S40 of FIG. 4, the musculoskeletal disorder prediction apparatus 100 may determine a pre-rated musculoskeletal disorder risk rating based on the updated range of motion. In particular, the musculoskeletal disorder prediction apparatus 100 may predict a change time of the determined risk rating based on at least one distance and/or angle change information extracted.

The above-described predetermined trajectory guide may be included in the musculoskeletal disorder prediction model according to the exemplary embodiment of the present disclosure.

Meanwhile, according to another exemplary embodiment of the present disclosure, the compensation movement may also be determined by tracking an interval of the same time unit movement trajectory of the second feature point B in addition to the method for determining the compensation movement through determination of trajectories of a plurality of feature points.

Referring to FIG. 7, the musculoskeletal disorder prediction apparatus 100 may determine the compensation movement interval on the movement trajectory of the user body part motion matched with the test motion based on rotational angular speed and/or angular acceleration information of the body part motion. The musculoskeletal disorder prediction apparatus 100 may determine the musculoskeletal range of motion in step S30 of FIG. 4 based on a determination result of the compensation movement interval.

The motion 620 referenced in FIGS. 6 and 7 indicates an example of a motion in which there is a limit in the range of motion of the shoulder musculoskeletal range of motion among the body part motions of the user.

The musculoskeletal disorder prediction apparatus 100 may split the movement trajectory in which the second feature point rotates around the extracted center point into a plurality of predetermined same-time unit intervals.

The motion 620 may be split into an interval 721, an interval 722, and an interval 723 in which the second feature point rotates and moves for the same time.

The musculoskeletal disorder prediction apparatus 100 may identify an interval which deviates from a predetermined trajectory guide based on the musculoskeletal test motion among the split intervals. The musculoskeletal disorder prediction apparatus 100 may determine an interval identified as deviating from the predetermined trajectory guide as the compensation movement interval.

Here, as described above, the predetermined trajectory guide as information on the movement trajectory of each reference feature point includes information which becomes a reference for the movement trajectory of the feature point extracted from the user image corresponding to the reference feature point.

According to an exemplary embodiment, the information which becomes the reference for the movement trajectory may include the number of intervals of spitting the movement trajectory of the feature point which rotates according to the test motion of the normal range of motion, and movement angular speed and/or angular speed change amount information of the feature point for each of the split intervals.

Referring to the motion 620 of FIG. 7, the musculoskeletal disorder prediction apparatus 100 may perform interval sampling at the same time interval based on the predetermined trajectory guide, and split the movement trajectory into the interval 721, the interval 722, and the interval 723 which are a total of three intervals.

As one example, the motion 610 may indicate the normal range of motion, the motion 610 may be one scene of the reference image, and the motion 620 may be one scene of the body part motion of the user.

To this end, the musculoskeletal disorder prediction apparatus 100 may extract the motion 610 and the reference feature point configuring the motion 610 from the reference image showing the normal range of motion in advance, and split the movement trajectory of the feature point into the interval 711, the interval 712, and the interval 713 based on a time required for entire rotating movement in the normal range of motion.

In an exemplary embodiment, the musculoskeletal disorder prediction apparatus 100 may acquire time information required for rotating movement for each interval, and pre-store angular speed information for each interval as trajectory guide information.

In another exemplary embodiment, the musculoskeletal disorder prediction apparatus 100 may detect a change amount of an angular speed in which the feature point rotates on a boundary of each interval, and pre-store angular acceleration information at a first interval end point and a second interval start point as trajectory guide information.

In addition, the trajectory guide may store the stored angular speed and angular acceleration as information which becomes a reference, and an error allowable range compared to the guide may be predetermined.

The musculoskeletal disorder prediction apparatus 100 may compare each interval of the motion 610 and each interval of the motion 620 by using the trajectory guide implemented in advance as described above, and determine, as the compensation movement interval, an interval which deviates from the predetermined trajectory guide based on the test motion among the split intervals.

The musculoskeletal disorder prediction apparatus 100 excludes at least a part of the compensation movement interval from the range of motion determined in step S30 of FIG. 4 to update the range of motion.

When described with reference to FIG. 8, the musculoskeletal disorder prediction apparatus 100 may determine the interval which deviates from the trajectory guide as the compensation movement interval, and when a trajectory in an opposite direction to the trajectory guide of the second feature point B is extracted, the musculoskeletal disorder prediction apparatus 100 may determine that there is the limit in the range of motion.

As a result, the musculoskeletal disorder prediction apparatus 100 may determine a first position at which the angular speed of the rotating movement trajectory becomes 0 as a start point of the compensation movement interval.

For example, as the user body part motion may be performed by the movement trajectory 811, the feature point B rotatably moves upwards, and a position at a moment when the angular speed becomes 0 according to the limit of the range of motion may be determined as the start point of the compensation movement interval.

After the angular speed initially becomes 0, the body part motion is additionally continued by the compensation movement, so an upward movement trajectory may appear partially. The musculoskeletal disorder prediction apparatus 100 may determine a second position at which the angular speed becomes 0 again due to the limit of the compensation movement while showing the upward movement trajectory as such, as an end point of the compensation movement interval.

The musculoskeletal disorder prediction apparatus 100 may determine the compensation movement interval based on the start point and the end point.

Up to now, the exemplary embodiment of determining the musculoskeletal range of motion considering the compensation movement is primarily described in performing the predetermined test motion, but the exemplary embodiment of the present disclosure is not limited thereto. According to yet another exemplary embodiment of the present disclosure, the compensation movement which influences one musculoskeletal range of motion may also influence another musculoskeletal range of motion. Subsequently, a method for predicting the musculoskeletal disorder risk considering an influence which the compensation movement exerts on a complex musculoskeletal range of motion is described.

After the user performs the test motion for the musculoskeletal system, an additional body part motion may be acquired by the camera 51 of FIG. 1.

The musculoskeletal disorder prediction apparatus 100 may identify the acquired additional body part motion, and determine whether the identified body part motion corresponds to a secondary test motion for the musculoskeletal system which becomes the target of the test motion described above.

In an exemplary embodiment, the musculoskeletal disorder prediction apparatus 100 may also determine a range of motion of a musculoskeletal system which performs the additional body part motion based on the identified additional body part motion.

The musculoskeletal disorder prediction apparatus 100 may determine whether there is a feature point overlapped with the feature point extracted when diagnosing a predetermined test motion, primary test motion, among the plurality of feature points extracted from the user image by performing the additional body part motion.

For example, the musculoskeletal disorder prediction apparatus 100 may identify that at least one feature point of the third feature point X1 and the fourth feature point Y1 is also extracted while performing the additional body part motion matched with the secondary test motion.

When at least one change information which deviates from the predetermined trajectory guide is extracted based on the secondary test motion, of distance change information and angle change information generated for at least one extracted feature point, the musculoskeletal disorder prediction apparatus 100 may identify at least one feature point of the third feature point and the fourth feature point as a feature point where the compensation movement occurs redundantly.

In step S40 of FIG. 4, the musculoskeletal disorder prediction apparatus 100 may determine a pre-rated musculoskeletal disorder risk rating based on the determined range of motion.

In an exemplary embodiment, the musculoskeletal disorder prediction apparatus 100 may determine a risk weight based on at least one extracted change information, and also predict a change time of the musculoskeletal risk rating of the user based on the determined risk weight.

Next, in order to determine the influence which the compensation movement influencing one musculoskeletal range of motion exerts on another musculoskeletal range of motion, according to yet another exemplary embodiment of the present disclosure, the musculoskeletal disorder prediction apparatus 100 may match the straight line 800 of FIG. 8 with a skeleton portion among elements constituting the musculoskeletal system, match the muscle area X including the identified third feature point with a first muscle portion among factors of the musculoskeletal system, and match the muscle area Y including the identified fourth feature point with a second muscle portion among the factors of the musculoskeletal system.

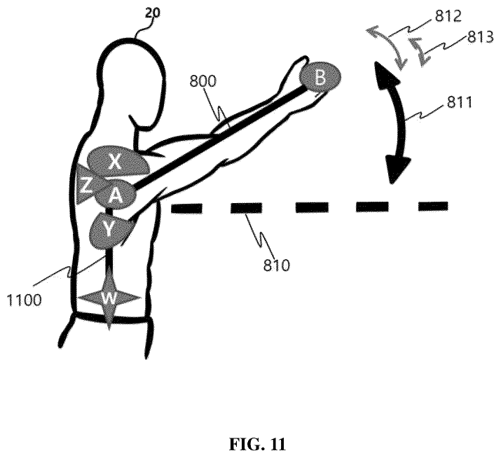

Referring to FIG. 11, the musculoskeletal disorder prediction apparatus 100 may extract a feature point of a body part including another musculoskeletal system connected to at least one element of the skeleton portion, the first muscle portion, and the second muscle portion. In FIG. 11, as examples of at least some factors of another musculoskeletal system, a skeleton 1100, a muscle area Z, and a tissue area W are illustrated.

The musculoskeletal disorder prediction apparatus 100 may set an area including the feature point for another musculoskeletal system as a compensation movement spread area. In the above description, the muscle area including at least one of the feature point X1 and the feature point Y1 referenced in FIG. 9 is the area where the compensation movement occurs, and the compensation movement may indirectly influence an area connected or contacted to the area where the compensation movement occurs.

That is, when the skeleton corresponding to the straight line 800 rotates, the movement of the muscle occurs, such as shape transformation of the muscle area X and/or the muscle area Y by the compensation movement, so the error may occur in measuring the musculoskeletal range of motion, and additionally, the error in measuring the range of motion may occur by muscle movement or support power of the muscle area Z and the tissue area W connected or contacted to the muscle area X and/or the muscle area Y.

According to an exemplary embodiment, the musculoskeletal disorder prediction apparatus 100 may consider a compensation movement area as an error cause of the measurement of the musculoskeletal range of motion, and furthermore, may also consider the compensation movement spread area as the error cause of the musculoskeletal range of motion, and reflect the compensation movement spread area to the determination of the range of motion and the prediction of the risk.

FIG. 12 illustrates an example in which the user's body part motion of FIG. 11 is expressed as a feature point connection structure as in FIG. 9. In particular, the feature connection structure is illustrated as a structure in which each of a feature point Z1 and a feature point W1 included in the muscle area Z and the tissue W adjacent to the center point A is connected to the center point A indicating the center point of the shoulder skeleton in straight lines 1200 and 1100.

Referring to FIG. 12, the musculoskeletal disorder prediction apparatus 100 may identify a body part motion corresponding to another musculoskeletal test motion other than the previously tested musculoskeletal system among the pre-registered musculoskeletal test motions in the user image by using the motion recognition model implemented in advance.

For example, in FIG. 12, the compensation movement may influence at least one of the feature point Z1 connected to the feature point X1 and the feature point W1 connected to the center point A by the straight line 1100 contacted to the feature point Y1, and the musculoskeletal disorder prediction apparatus 100 may set the skeleton 1100, the muscle area Z, and the tissue area W which belong to another musculoskeletal system connected to the musculoskeletal system in which the test motion is performed as the compensation movement spread area.

The musculoskeletal disorder prediction apparatus 100 may determine the range of motion of another musculoskeletal system based on the corresponding body part motion, and also predict the disorder risk of another musculoskeletal system based on information on the determined range of motion and compensation movement spread area by using the musculoskeletal disorder prediction model implemented in advance.

According to yet another exemplary embodiment of the present disclosure, the musculoskeletal disorder prediction apparatus 100 may also predict the disorder risk of another musculoskeletal system based on a result of predicting the musculoskeletal disorder risk and information on the compensation movement spread area.

According to the exemplary embodiments of the present disclosure described above, in order to predict the musculoskeletal disorder risk, the musculoskeletal disorder prediction apparatus 100 may implement the prediction model in advance. Hereinafter, the musculoskeletal disorder prediction apparatus 100 in the exemplary embodiment of predicting the musculoskeletal risk may be called the musculoskeletal disorder prediction model implementation apparatus 100 in the exemplary embodiment of implementing the prediction model, and may be abbreviated as the prediction model implementation apparatus 100.

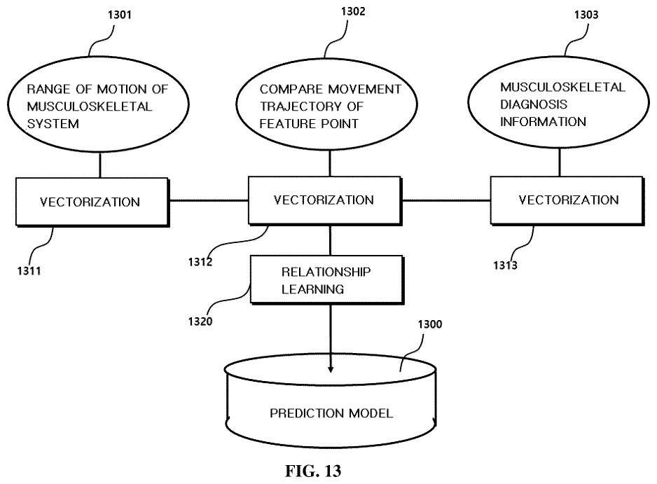

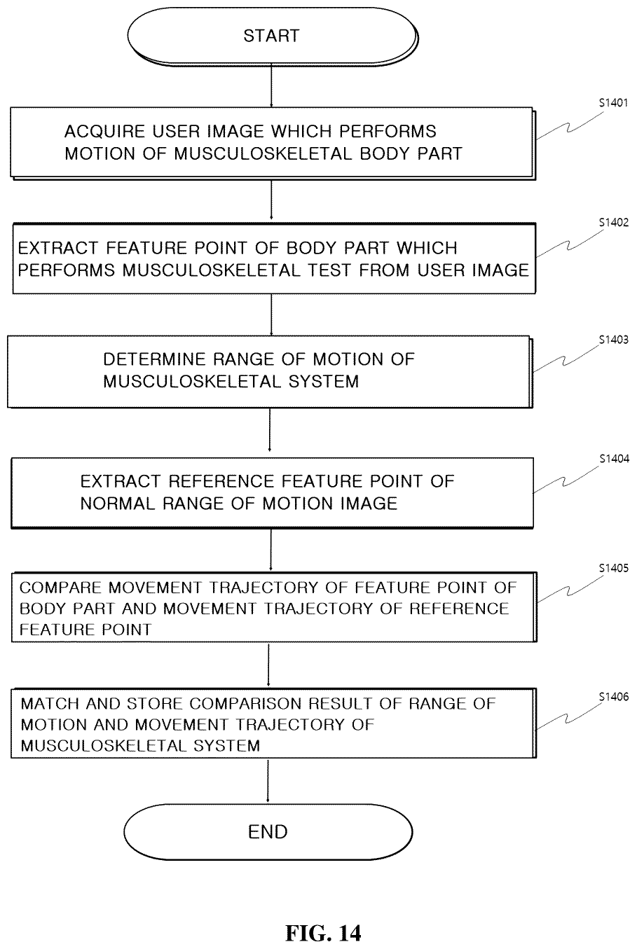

Referring to FIGS. 13 and 14, the prediction model implementation exemplary embodiment of the prediction model implementation apparatus 100 is described, and a duplicated description with the musculoskeletal risk prediction exemplary embodiment may be omitted.

FIG. 13 is a conceptual diagram of a prediction model according to still yet another exemplary embodiment of the present disclosure, and FIG. 14 is a flowchart of a method for implementing the prediction model of FIG. 13. Each step of FIG. 14 may be implemented by the prediction model implementation apparatus 100.

Referring to FIG. 14, the prediction model implementation apparatus 100 may acquire a user image in which a pre-registered musculoskeletal test motion is performed, in S1401.

The prediction model implementation apparatus 100 may extract a feature point of a body part which performs a musculoskeletal test motion from the user image by using a motion recognition model implemented in advance, in S1402. Unlike the risk prediction exemplary embodiment, in the case of the prediction model implementation exemplary embodiment, it is not necessary to determine whether the body part motion in the user image matches the test motion, and in the user's body part motion, the feature point may be extracted by determining that a specific test motion should be performed.

The prediction model implementation apparatus 100 may determine a range of motion ROM of a musculoskeletal system corresponding to the body part based on a movement trajectory of the extracted feature point of the body part, in S1403.

The prediction model implementation apparatus 100 may extract a reference feature point of a pre-registered musculoskeletal normal range of motion image, in S1404.

Hereinafter, the prediction model implementation apparatus 100 may extract the reference feature point of the normal range of motion image by a similar scheme to the feature point extraction exemplary embodiment for predicting the musculoskeletal disorder risk in the above description.

The prediction model implementation apparatus 100 may identify a plurality of feature points of the body part including the musculoskeletal system in the pre-registered musculoskeletal normal range of motion image by using the motion recognition model implemented in advance, and identify a first feature point included in a first area and a second feature point included in a second area at both distal ends of the body part among them. For example, the first feature point A and the second feature point B at both distal ends of the straight line 800 of FIG. 8 corresponding to the skeleton portion may be identified.

Specifically, the prediction model implementation apparatus 100 may set the first feature point which becomes a center of rotating movement as the center point, and the prediction model implementation apparatus 100 may also identify the third feature point X1 and the fourth feature point Y1 referenced in FIG. 9.

In the above description, in the musculoskeletal risk prediction exemplary embodiment, user motions in FIGS. 8 and 9 are the motions for testing the user's musculoskeletal disorder, and the extracted feature point is also for measuring the range of motion and generating the compensation movement information, but in the prediction model implementation exemplary embodiment, the user motions illustrated in FIGS. 8 and 9 are the test motions performed to implement the prediction model, and the extracted feature point should be appreciated as data generated to implement the prediction model.

The prediction model implementation apparatus 100 may extract target feature points matched with the first feature point, the second feature point, the third feature point, and the fourth feature point, respectively among the extracted feature points of the body part.

The prediction model implementation apparatus 100 may compare the movement trajectory of the extracted feature point of the body part and a movement trajectory of a reference feature point, in S1405.

In an exemplary embodiment, the prediction model implementation apparatus 100 may first-split the movement trajectory of the feature point of the body part into a plurality of predetermined same time-unit intervals, and similarly, second-split the movement trajectory of the reference feature point into a plurality of predetermined same time-unit intervals.

The prediction model implementation apparatus 100 may first-identify distance change information and angle change information of the feature point extracted from each first-split interval, and second-identify distance change information and angle change information of the reference feature point for each second-split interval.

According to an exemplary embodiment of the present disclosure, the prediction model implementation apparatus 100 may match and compare the first identified information and the second identified information for each interval.

In an exemplary embodiment, the prediction model implementation apparatus 100 may first-compare the movement trajectories of the first feature point A and the second feature point B, and movement trajectories of target feature points matched therewith, respectively.

In another exemplary embodiment, the prediction model implementation apparatus 100 may also second-compare movement trajectories of the third feature point X1 and the fourth feature point Y1, and movement trajectories of target feature points matched with the third feature point X1 and the fourth feature point Y1, respectively.

In order to determine the compensation movement, the prediction model implementation apparatus 100 may determine whether a difference between the first identified information and the second identified information exceeds a predetermined range according to a first comparison result. When the difference exceeds the predetermined range according to the determination result, the prediction model implementation apparatus 100 may determine that the compensation movement occurs in a matching interval in which the excess is confirmed.

Specifically, the prediction model implementation apparatus 100 may determine that the compensation movement occurs when a difference for at least one of a distance and an angle of the movement trajectory exceeds a predetermined range according to a result of comparing the movement trajectories of the first feature point and the second feature point, and the movement trajectories of the target feature points matched therewith, respectively.

Further, the prediction model implementation apparatus 100 may also perform comparison of the movement trajectories of the target feature points matched with the third feature point and the fourth feature point, respectively among the extracted feature points of the body part, and determination of the occurrence of the compensation movement in a similar scheme.

Referring back to FIG. 14, according to an exemplary embodiment of the present disclosure, the prediction model implementation apparatus 100 may match and store the comparison results of the determined range of motion and movement trajectory, in S1406.

In an exemplary embodiment, the prediction model implementation apparatus 100 may also match and store the determined range of motion, the first compared result, and the second compared result.

Meanwhile, according to still yet another exemplary embodiment of the present disclosure, the prediction model implementation apparatus 100 may collect user's musculoskeletal diagnosis information including a response to a pain level of the user by performing the pre-registered musculoskeletal test motion.

Referring to FIG. 13, the prediction model implementation apparatus 100 may also match and store a range of motion 1301, a movement trajectory comparison result 1302, and user's musculoskeletal diagnosis information 1303.

In an exemplary embodiment, the prediction model implementation apparatus 100 embeds the range of motion 1301 and the movement trajectory comparison result 1302 which are matched and stored for each predetermined cycle to generate a first vector value set, 1311 and 1312.

In an exemplary embodiment, the prediction model implementation apparatus 100 also embeds the user's musculoskeletal diagnosis information, 1303, to generate a second vector value set, 1313.

In an exemplary embodiment, the prediction model implementation apparatus 100 may learn a relationship between the first vector value set and the second vector value set, 1320.

The prediction model implementation apparatus 100 may generate a musculoskeletal disorder prediction model 1300 based on the learning of the relationship between the first vector value set and the second vector value set.

In an exemplary embodiment, as a latest range of motion for the user's musculoskeletal system is determined, the prediction model implementation apparatus 100 may generate the user's musculoskeletal diagnosis information based on the determined latest range of motion by using the generated musculoskeletal disorder prediction model. Further, the prediction model implementation apparatus 100 may also predict the user's musculoskeletal disorder risk based on the generated diagnosis information.

When collecting the user's musculoskeletal diagnosis information, the prediction model implementation apparatus 100 may also generate a text-based vector value by performing a text analysis for a predetermined item among the collected diagnosis information.

Further, the prediction model implementation apparatus 100 may also generate an image-based vector value by extracting a property of the user from the user image by using an implemented analysis model.

The prediction model implementation apparatus 100 compares the text-analyzed vector value and the image-based vector value for the property extracted from the image, and when there is a difference which exceeds a predetermined range, the prediction model implementation apparatus 100 may generate a text based on the extracted image-based vector value.

The prediction model implementation apparatus 100 may set the text generated in the predetermined item. Through this, when there is an error in the collected diagnosis information to which a user survey, etc. is reflected, the prediction model implementation apparatus 100 generates and applies a property-based text extracted from the user image to correct the error of the diagnosis information.

According to still yet another exemplary embodiment of the present disclosure, the prediction model implementation apparatus 100 may generate an exercise program matched with the risk predicted for the musculoskeletal system, and provide the generated exercise program through the user terminal 300.

Here, the exercise program may include exercise guide information for the area X including the third feature point X1 and the area Y including the fourth feature point Y1 in which the compensation movement for the body part motion occurs. Further, the exercise guide information which is for alleviating the compensation movement may include information for guiding a motion opposite to the motion in which the compensation movement occurs.

The determination and/or computation methods of the processor according to the exemplary embodiments of the present disclosure described with reference to the accompanying drawings so far can be performed by executing a computer program implemented in computer-readable code. The computer program may be transmitted from a first computing apparatus to a second computing apparatus through a network such as the Internet and installed in the second computing apparatus to be used in the second computing apparatus. The first computing apparatus and the second computing apparatus include all of a server apparatus, a fixed computing apparatus such as a desktop PC, and a mobile computing apparatus such as a laptop, a smart phone, and a tablet PC.

Hereinabove, the exemplary embodiments of the present disclosure have been described with the accompanying drawings, but it can be understood by those skilled in the art that the present disclosure can be executed in other detailed forms without changing the technical spirit or requisite features of the present disclosure. Therefore, it should be appreciated that the aforementioned exemplary embodiments are illustrative in all aspects and are not restricted.

What is claimed is:

1. A method for implementing a musculoskeletal disorder prediction model, which is performed by a musculoskeletal disorder prediction model implementation apparatus, the method comprising:
acquiring a user image which performs a pre-registered musculoskeletal test motion;
extracting a feature point of a body part which performs the musculoskeletal test motion from the user image by using a motion recognition model implemented in advance;
determining a range of motion ROM of a musculoskeletal system corresponding to the body part based on a movement trajectory of the extracted feature point of the body part;
extracting a reference feature point of a pre-registered musculoskeletal normal range of motion image;
comparing the movement trajectory of the extracted feature point of the body part and a movement trajectory of the reference feature point; and
matching and storing a comparison result of the determined range of motion and the movement trajectory.

2. The method according to claim 1, wherein the comparing of the movement trajectory of the extracted feature point of the body part and the movement trajectory of the reference feature point includes
first-splitting the movement trajectory of the feature point of the body part into a plurality of predetermined same time-unit intervals,
second-splitting the movement trajectory of the reference feature point into the plurality of predetermined same time-unit intervals,
first-identifying distance change information and angle change information of the first-split interval-specific extracted feature points,
second-identifying distance change information and angle change information of the second-split interval-specific extracted reference feature points, and
matching and comparing the first-identified information and the second-identified information for each interval.

3. The method according to claim 2, wherein the matching and comparing of the first-identified information and the second-identified information for each interval includes
when a difference between the first-identified information and the second-identified information exceeds a predetermined range according to the comparison result, determining that a compensation movement occurs in a matching interval in which the excess is confirmed.

4. The method according to claim 3, wherein the extracting of the reference feature point includes
identifying a plurality of feature points of the body part including the musculoskeletal system in the pre-registered musculoskeletal normal range of motion image by using the motion recognition model implemented in advance, and
identifying a first feature point included in a first area and a second feature point included in a second area at both distal ends of the body part among the plurality of feature points, and
the comparing of the movement trajectory of the extracted feature point of the body part and the movement trajectory of the reference feature point includes
setting the first feature point which becomes a center of a rotating movement as a center point, and extracting target feature points matched with the first feature point and the second feature point, respectively among the extracted feature points of the body part, and
first-comparing movement trajectories of the first feature point and the second feature point, and movement trajectories of the target feature points matched therewith, respectively.

5. The method according to claim 4, wherein the extracting of the reference feature point includes
identifying a third feature point forming a first angle based on a straight line including the first feature point and the second feature point within a predetermined first distance range from the first feature point, and
identifying a fourth feature point forming a second angle based on the straight line within a predetermined second distance range from the first feature point, and
the first-comparing of the movement trajectories of the first feature point and the second feature point, and the movement trajectories of the target feature points matched therewith, respectively includes
determining that a compensation movement occurs when a difference for at least one of a distance and an angle of the movement trajectory exceeds a predetermined range according to a result of comparing the movement trajectories of the first feature point and the second feature point, and the movement trajectories of the target feature points matched therewith, respectively.

6. The method according to claim 5, wherein the comparing of the movement trajectory of the extracted feature point of the body part and the movement trajectory of the reference feature point further includes
extracting target feature points matched with the third feature point and the fourth feature point, respectively among the extracted feature points of the body part,
the determining that the compensation movement occurs includes
second-comparing movement trajectories of the third feature point and the fourth feature point, and movement trajectories of target feature points matched with the third feature point and the fourth feature point, respectively, and
the matching and storing of the comparison result of the determined range of motion and the movement trajectory includes
matching and storing the determined range of motion, the first-compared result, and the second-compared result.

7. The method according to claim 1, wherein the matching and storing of the comparison result of the determined range of motion and the movement trajectory includes
collecting musculoskeletal diagnosis information of a user, which including a response to a pain level of the user by performing the pre-registered musculoskeletal test motion, and
matching and storing the range of motion, the comparison result of the movement trajectory, and the musculoskeletal diagnosis information of the user.

8. The method according to claim 7, wherein the matching and storing of the range of motion, the comparison result of the movement trajectory, and the musculoskeletal diagnosis information of the user includes
generating a first vector value set by embedding the matched and stored range of motion and the comparison result of the movement trajectory, and generating a second vector value set by embedding the musculoskeletal diagnosis information of the user, for each predetermined cycle, and
learning a relationship between the first vector value set and the second vector value set.

9. The method according to claim 8, comprising:
generating the musculoskeletal disorder prediction model based on the learning of the relationship between the first vector value set and the second vector value set;
generating, as a latest range of motion for the musculoskeletal system of the user is determined, the musculoskeletal diagnosis information of the user based on the determined latest range of motion by using the generated musculoskeletal disorder prediction model; and
predicting a disorder risk of the musculoskeletal system of the user based on the generated diagnosis information.

10. The method according to claim 7, wherein the collecting of the musculoskeletal diagnosis information of the user includes
generating a first vector value by performing a text analysis for a predetermined item in the collected diagnosis information,
generating a second vector value by extracting a property of the user from the user image by using an analysis model implemented in advance,
comparing the text-analyzed first vector value and the second vector value for the extracted property,
generating a text based on the extracted second vector value when there is a difference which exceeds a predetermined range according to the comparison result, and
setting the generated text in the predetermined item.

11. The method according to claim 9, further comprising:
generating an exercise program matched with a risk predicted for the musculoskeletal system; and
providing the generated exercise program through a terminal of the user,
wherein the exercise program includes exercise guide information for an area including the third feature point and an area including the fourth feature point where the compensation movement for the body part motion occurs.

12. An apparatus for implementing a musculoskeletal disorder prediction model, the apparatus comprising:
one or more processors;
a network interface receiving a user image including a musculoskeletal body part motion;
a memory loading computer programs executed by the processors; and
a storage storing the computer programs,
wherein the computer program includes
an operation of acquiring the user image which performs a pre-registered musculoskeletal test motion,
an operation of extracting a feature point of a body part which performs the musculoskeletal test motion from the user image by using a motion recognition model implemented in advance,
an operation of determining a range of motion ROM of a musculoskeletal system corresponding to the body part based on a movement trajectory of the extracted feature point of the body part,
an operation of extracting a reference feature point of a pre-registered musculoskeletal normal range of motion image,
an operation of comparing the movement trajectory of the extracted feature point of the body part and a movement trajectory of the reference feature point, and
an operation of matching and storing a comparison result of the determined range of motion and the movement trajectory.
